(19) United States
(12) Patent Application Publication
(10) Pub. No.: US 2026/0315228 A1
(43) Pub. Date: Oct. 15, 2026

(54) BONE-ANCHORED OSSEOINTEGRATION IMPLANT AND METHOD OF IMPLANTATION

(71) Applicant: ALM Ortho, Inc., Scarborough, ME (US)

(72) Inventor: Brian R. McLaughlin, Yarmouth, ME (US)

(21) Appl. No.: 19/648,174

(22) Filed: Apr. 15, 2026

Related U.S. Application Data
(60) Provisional application No. 63/789,075, filed on Apr. 15, 2025.

(51) Int. Cl. A61F 2/28; A61F 2/30

(52) U.S. Cl. CPC A61F 2/28; A61F 2002/30784; A61F 2002/3093; A61F 2002/30985; A61F 2220/0041; A61F 2310/00023; A61F 2310/00407; A61F 2310/0088

(57) ABSTRACT

A bone-anchored implant for osseointegration includes a main body having a proximal end, a distal end, and a longitudinal axis configured to conform to the anatomical curvature and shape of a patient's bone canal, providing a most optimal opportunity for initial fixation for early ambulation as well as the best opportunity for bone ingrowth.

### BACKGROUND INFORMATION

### Field of the Invention

The field of the present invention relates to bone-anchored implants and, more specifically, to an osseointegration implant that offers an enhanced patient-matched design, optimized osseointegration, single-stage surgical implantation, and a faster return to functional activity, including early loading and ambulation, along with the restoration of proprioception.

### Discussion of Prior Art

Bone-anchored implants have long been used in various medical fields, such as dental, orthopedic, and prosthetic applications. These implants typically rely on osseointegration, a process where the implant becomes integrated into the bone, providing a stable and durable interface. However, existing implants often face challenges related to suboptimal integration, long recovery times, and the need for multiple surgeries, especially when the implant is used for prosthetic applications or when it is intended to replace missing or damaged limbs.

A significant issue in current osseointegration devices is the generic, one-size-fits-all approach in design, leading to suboptimal outcomes for individual patients. The bone anatomy, shape, and structure of each patient vary, and as such, a standardized implant design may not provide the most effective fit or osseointegration, potentially increasing the risk of implant failure or complications.

Furthermore, many bone-anchored implants require multiple stages of surgery. The first stage typically involves the insertion of the implant and the attachment of a post to the bone, followed by a healing period during which the implant integrates with the bone. A second stage is then required to attach the external components, which prolongs recovery time and requires additional invasive procedures. This multi-stage surgical process can be burdensome to patients, increasing the risk of infection, complications, and delaying the return to normal functional activities, such as walking or bearing weight.

Another challenge with current bone-anchored implants is the delayed loading and ambulation times. Many implants require a lengthy healing period to allow for sufficient osseointegration before weight-bearing activities can be safely undertaken. As a result, patients experience extended periods of immobility, affecting their overall quality of life.

Additionally, the restoration of proprioception, or the body's ability to sense the position and movement of limbs, is critical for the proper function of prosthetic devices. In many cases, current bone-anchored implants fail to adequately restore proprioception, leading to difficulties with coordination, balance, and mobility, which can severely impact a patient's ability to adapt to a prosthetic limb.

Thus, there exists a need for an improved bone-anchored osseointegration implant that addresses these challenges. The present invention solves these problems by offering a patient-matched implant design, optimized for osseointegration, enabling single-stage surgery, earlier loading and ambulation, and the restoration of proprioception. These improvements provide significant benefits to patients, allowing for a faster recovery, enhanced implant stability, and a more natural and functional prosthetic experience.

### BRIEF SUMMARY OF THE INVENTION

The invention is a bone-anchored osseointegration implant that is customized for a particular patient's anatomy. The implant has a monolithic structure that follows the patient's anatomic curvature, with the device having an outer surface that is largely, but not necessarily entirely, in the form of a lattice structure that promotes bone growth.

A custom-designed broach is used to prepare the bone canal in a manner that will create a bone-to-bone connection and a press fit of the implant in the bone canal. The implant has a proximal taper for stress relief, and the majority of the implant has a porous titanium coating that is fully integrated with solid titanium stem. A distal taper region is coated with titanium nitride or a similar metal ceramic coating to enhance the surface condition of the implant. An endcap on the implant allows for ease of insertion and, generally, does not catch against the bone canal when being inserted.

The implant is suitable for any osseointegration procedure, and is particularly beneficial for use in the upper and lower extremities, particularly the humerus, radius, ulna, or a combination of radius and ulna, femur, and tibia. The customized shape may vary to match the particular bone canal, which helps to optimize the implant to bone contact which improves the overall fit and opportunity for rapid osseointegration.

The structure of the device allows for a comparatively easy insertion and optimal results as the precision fit of the implant along the anatomical curvature of the patient, with the press-fit of the lattice structure against the bone canal creating an ideal long-term integration of the implant into the bone.

### BRIEF DESCRIPTION OF THE DRAWINGS

The present invention is described with reference to the accompanying drawings. In the drawings, like reference numbers indicate identical or functionally similar elements. The drawings are not drawn to scale.

FIG. 1 is an anterior view of the implant inserted into a femur.

FIG. 2 is a lateral view of the implant inserted into a femur.

FIG. 3 is a cross-sectional view of FIG. 1.

FIG. 4 is a cross-sectional view of FIG. 2.

FIG. 5 is an anterior view of an embodiment of the implant.

FIG. 6 is a lateral view of an embodiment of the implant.

FIG. 7 is an anterior view of the implant.

FIG. 8 is a close-up view of structural ribs and lattice structure.

FIG. 9 is a cross-sectional view of the implant.

FIG. 10 is a close-up view of a portion of FIG. 9.

FIG. 11 is an isometric cross-sectional view for the device shown in FIGS. 7-10.

FIG. 12 is a close-up view of a portion of FIG. 11.

FIG. 13 is an exploded view of the connection assembly.

FIG. 14 is similar to FIG. 1 but illustrates the device used with the tibia.

FIG. 15 is similar to FIG. 2 but illustrates the device used with the tibia.

FIG. 16 is similar to FIG. 3 but illustrates the device used with the tibia.

FIG. 17 is similar to FIG. 4 but illustrates the device used with the tibia.

FIG. 18 is similar to FIG. 5 but illustrates one variant designed for use with the tibia.

FIG. 19 is a close-up view of a portion of FIG. 18.

FIG. 20 is a isometric view of the device shown in 18 with the rounded top removed.

FIG. 21 is a partial close-up view of the illustration in FIG. 20

FIG. 22 illustrates an embodiment of the device having an isogrid rib pattern.

FIG. 23 is a partial close-up of the embodiment shown in FIG. 22.

FIG. 24A is a illustration of a first patient-matched bone implant.

FIG. 24B is a illustration of a second patient-matched bone implant.

FIG. 24C is a illustration of a third patient-matched bone implant.

FIG. 24D is a illustration of a fourth patient-matched bone implant.

FIG. 24E is a illustration of a fifth patient-matched bone implant.

FIG. 24F is a illustration of a sixth patient-matched bone implant.

FIG. 24G is a illustration of a seventh patient-matched bone implant.

FIG. 24H is an illustration of an eighth patient-matched bone implant.

FIG. 25 is an anterior view of a broach designed for use with a femur.

FIG. 26 is a lateral view of the broach shown in FIG. 25.

FIG. 27 is a close-up view of a portion of the broach shown in FIG. 26.

FIG. 28 is a broach designed for use with a tibia.

FIG. 29 is a second variant of broach designed for use with the tibia.

FIG. 30 is a third variant of a broach designed for use with the tibia.

FIG. 31 is a partial close-up view of the broach shown in FIG. 30.

### DETAILED DESCRIPTION OF THE INVENTION

The present invention will now be described more fully in detail with reference to the accompanying drawings, in which the preferred embodiments of the invention are shown. This invention should not, however, be construed as limited to the embodiments set forth herein; rather, they are provided so that this disclosure will be complete and will fully convey the scope of the invention to those skilled in the art.

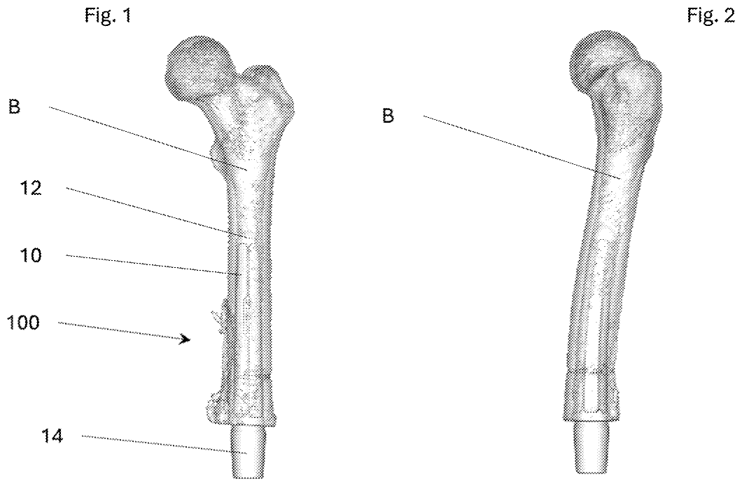

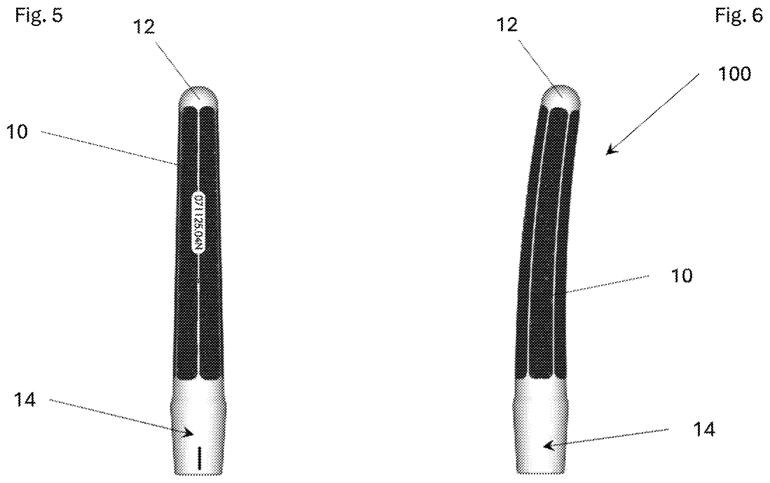

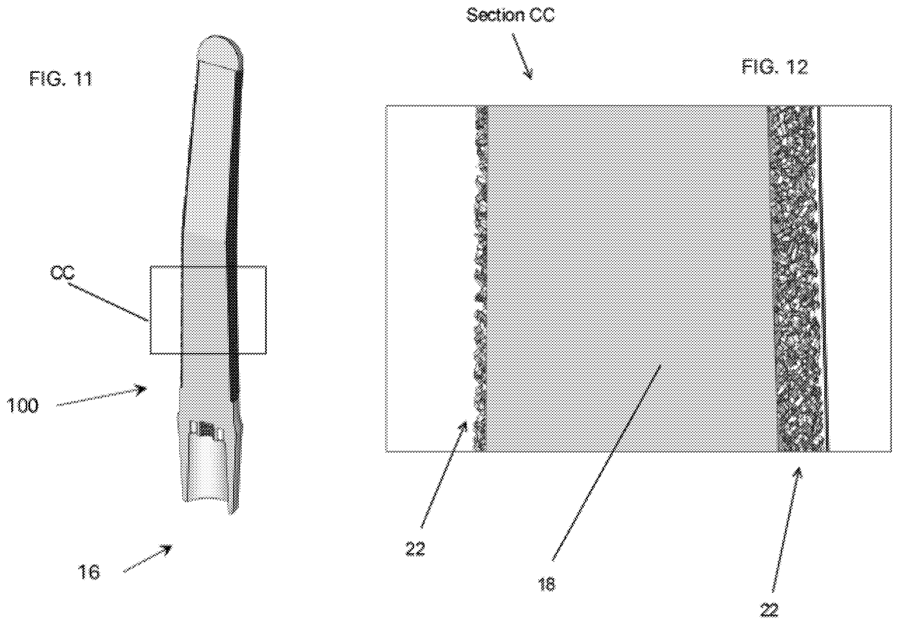

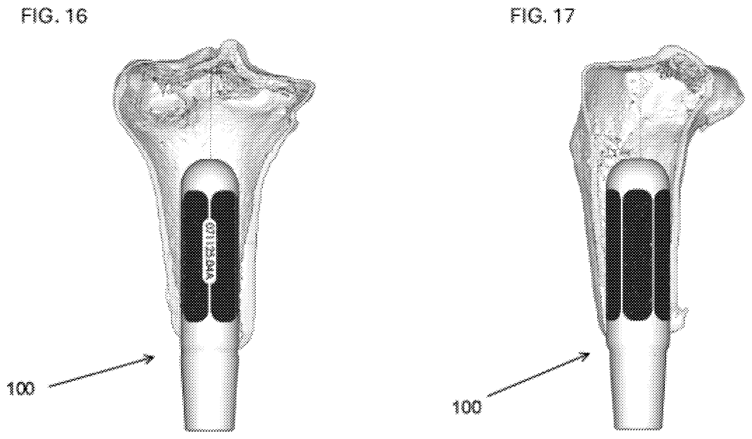

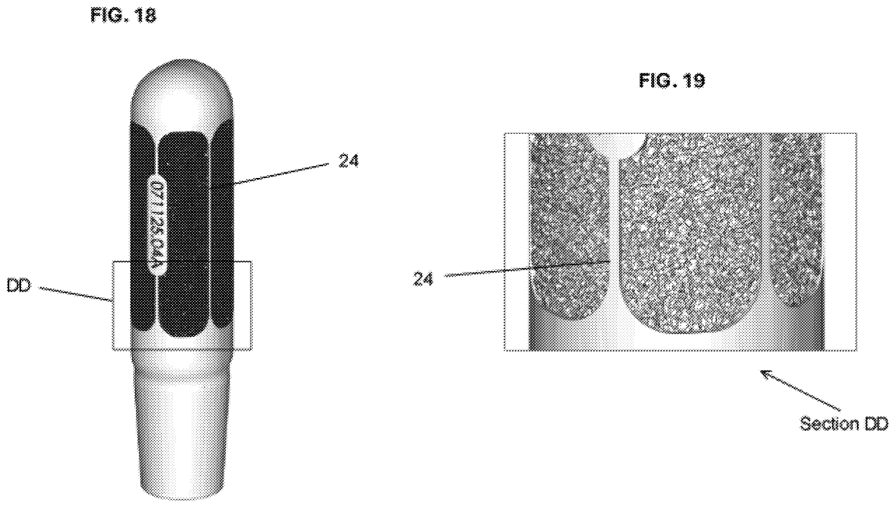

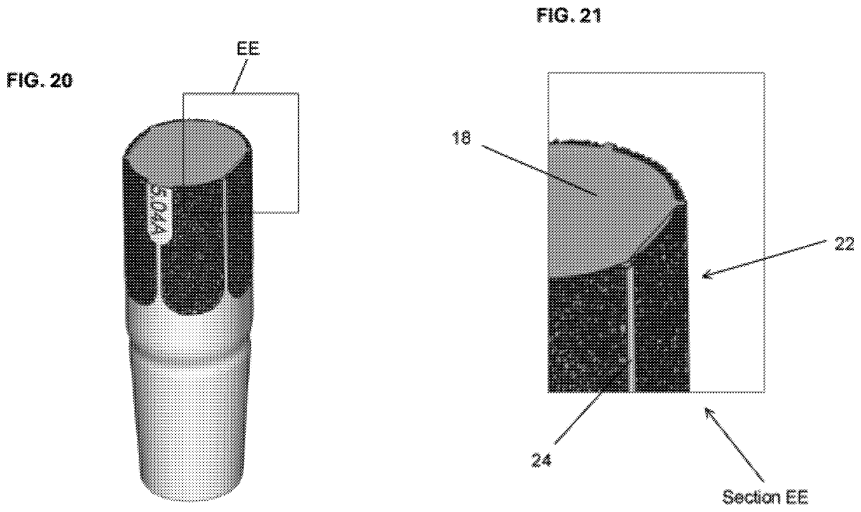

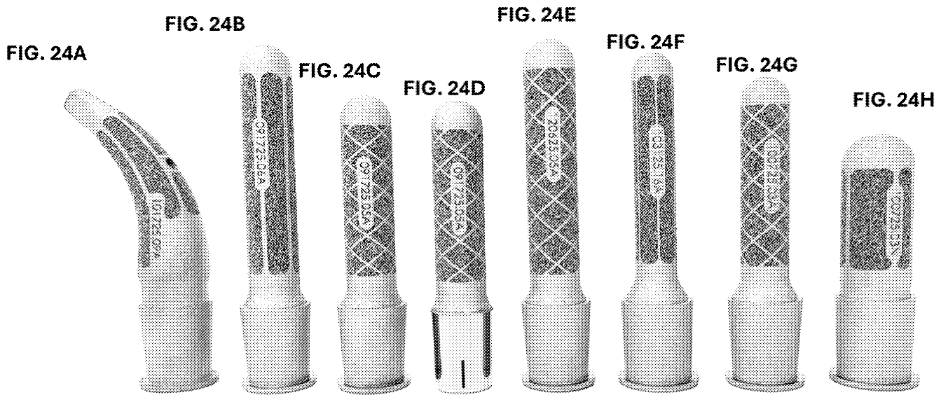

FIGS. 1-24H illustrate a patient-matched bone-anchored implant 100 for osseointegration, according to the invention. The implant 100 is configured to be inserted into a patient's bone canal C, after proper preparation of the canal, where it follows the anatomical curvature of the bone B. The implant is suitable for insertion into a number of different bones, for example, the femur, tibia, humerus, ulna and/or radius. In general, the inventive implant 100 may be fully inserted through a single-stage surgical procedure, and where the patient time to loading is on average as low as six to eight weeks.

The implant 100 includes a main body 10 that is a largely cylindrical in shape, with variations if needed to match the shape of the particular bone, a proximal end 12 that is at least slightly rounded so as to ease insertion into a bone canal. The proximal end 12 include may also include a taper region that is coated with a titanium nitride or similar substance, which enhances the surface condition for insertion into the prepared bone canal. The implant further includes a distal end 14, the distal end being configured to couple to a connector assembly 50 that attaches the implant 100 to a conventional prosthetic. The distal end 14 of the implant 100 may be machined smooth with a TiN (gold) coating for improved hardness for better wear resistance.

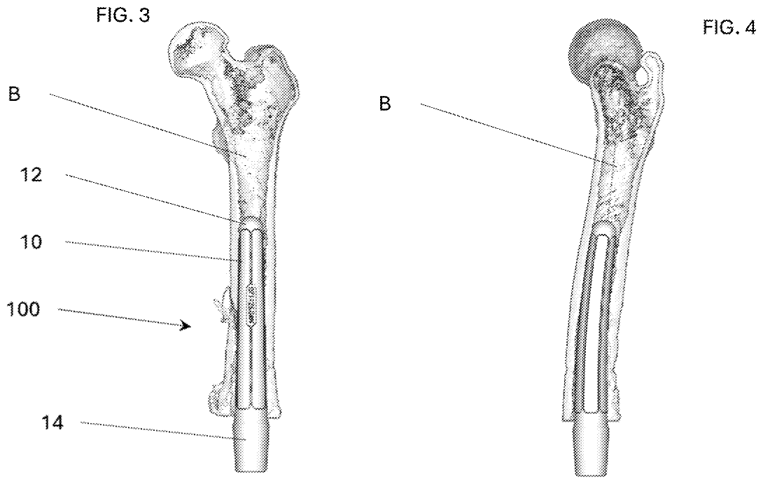

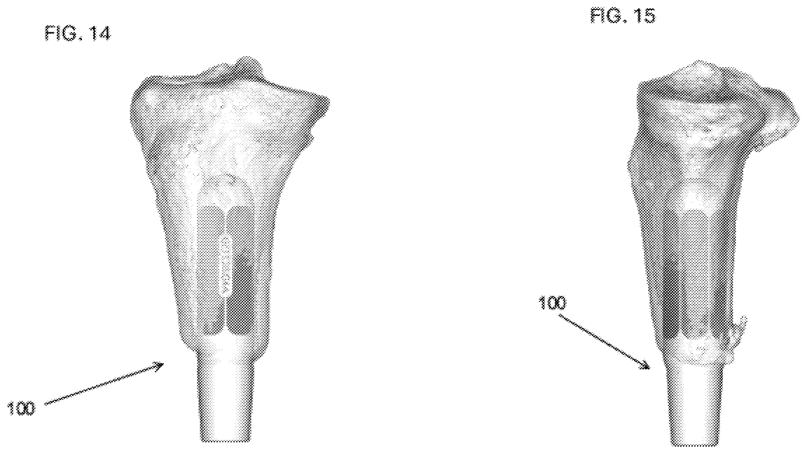

For example, FIGS. 1 and 2 illustrate an image of a transfemoral monolithic patient-matched bone-anchored implant 100, overlayed in the anatomy, with FIGS. 3 and 4 illustrating a cross-sectional view of the bone to better illustrate the implant 100. FIGS. 14-17 illustrating a similar implant 100 that is configured for a transtibial monolithic patient-matched bone-anchored implant.

The main body 10 is generally comprised of a 3D printed metal having a 3-dimensional porous coating. For example, it may be a porous titanium coating that is fully integrated with a titanium stem, creating a monolithic structure with surface area that has a lattice structure that is ideal for integrated bone growth. More specifically, the main body 10 may have a solid core 18, with a porous coating 22 having a thickness that ranges from 0.5 mm to 1.5 mm and also have a range of pore size from 400-800 microns, and is ideally around a 1 mm 3D porous coating with 500-micron pore size, which is optimal for bone ingrowth in an osseointegration process. In one preferred embodiment, the solid core 18 of the main body 10 of the implant 100 has a 1 mm thick 3D porous coating with 500-micron pore size for optimal bone ingrowth/osseointegration.

As noted, the device is 3d printed to fit a particular patient, and as a result the specific dimensions are likely to vary from one instance to another. For example, the embodiment shown in FIGS. 1-4 is designed for a specific patient's femur, and the main body 10 from the proximal end 12 to the distal end 14 has a length and shape that most effectively matches the shape of the bone. As a non-limiting example, measurements for a device matched for a femur may be on the order of 110 to 120 millimeters in length with diameters measuring between 15 and 18 millimeters, but again, specific measurements depend on the patient's specific bone structure and in that regard actual dimensions may be larger or smaller as determined by those skilled in the art. Different patients require different dimensions, and in another embodiment the main body may have, for instance, a length that is shorter and a diameter or shape that is different.

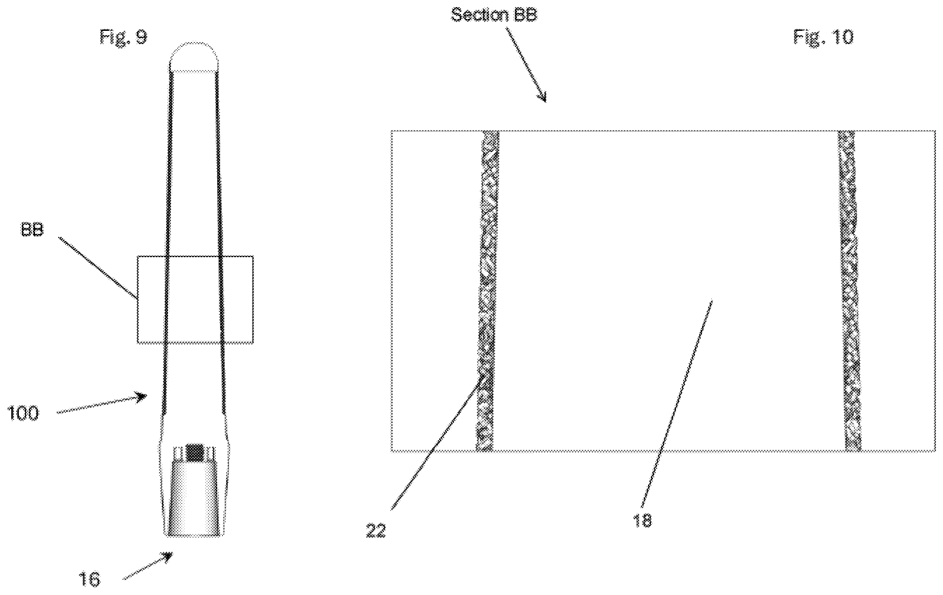

FIGS. 9-12 illustrate a constructional view of the implant, with FIGS. 10 and 12 highlighting the lattice and the solid substrate and how they are one, i.e. the monolithic structure, where they are created at the same time through the manufacturing process.

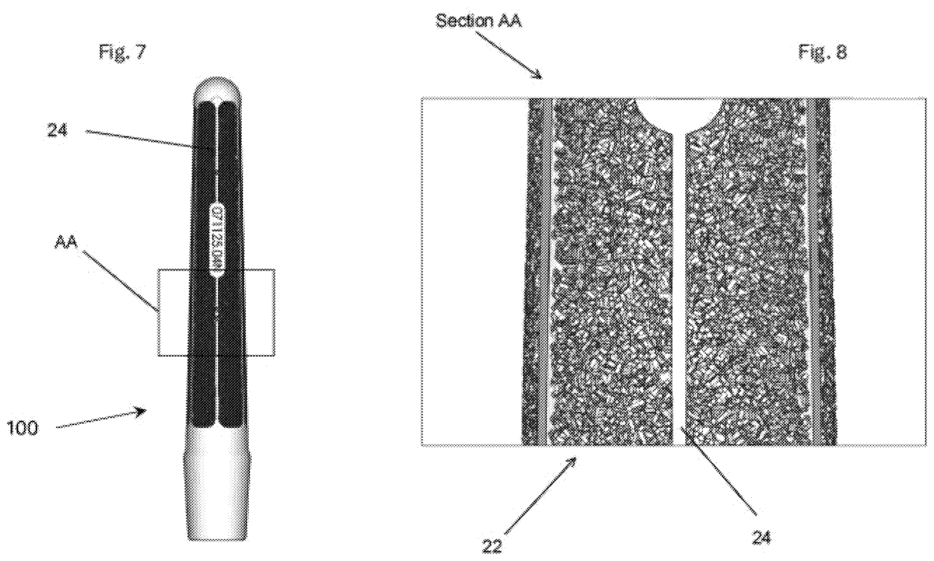

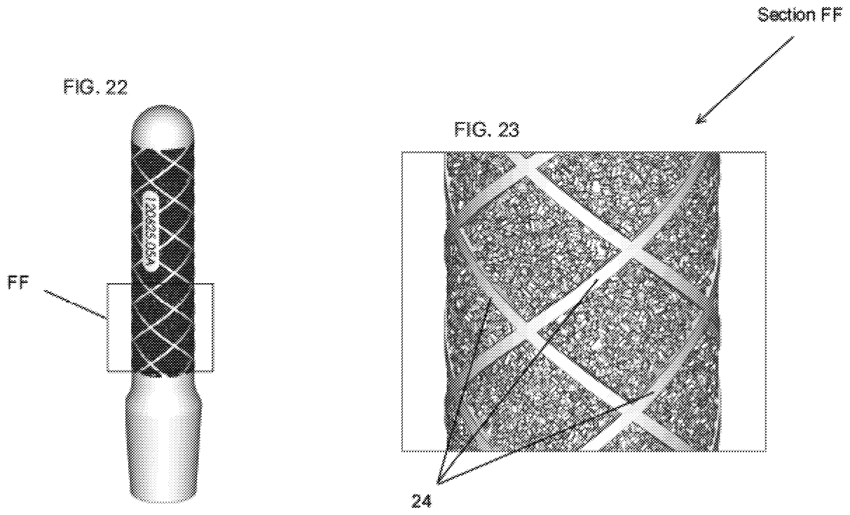

FIGS. 7, 8, and 17-23 illustrate structural ribs 24 that may be incorporated to strengthen the implant 100, and in particular such ribs may help to strengthen the implant when the bone is undergoing an activity that involves bending. In FIG. 22, The rib features are part of the solid substrate, whereas the lattice falls in between the isogrid features but are still manufactured at the same time and through the same process.

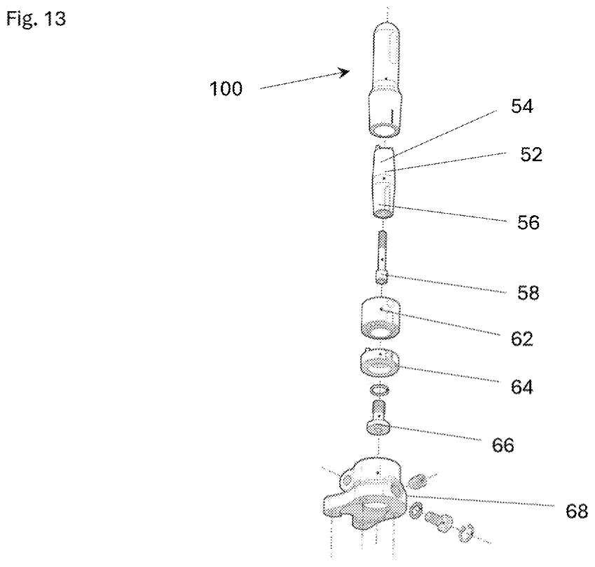

FIG. 13 illustrates one manner in which the implant 10 may be connected to the connector assembly 50. A dual cone 52 has proximal end 54 that is adapted to fit inside an opening 16 in the distal end 14 of the implant 100, and a threaded fastener 58, for example a locking screw, passes through the cone 52 to secure the cone in the implant 10. A distal end 56 of the dual cone fits in taper sleeve 62 and bushing 64 which are secured with a second fastener 66, for example, a locking screw, with the sleeve 62 and busing 64 fitting into the connector 68. The connector assembly 50 is configured to connect to a prosthetic in a conventional manner.

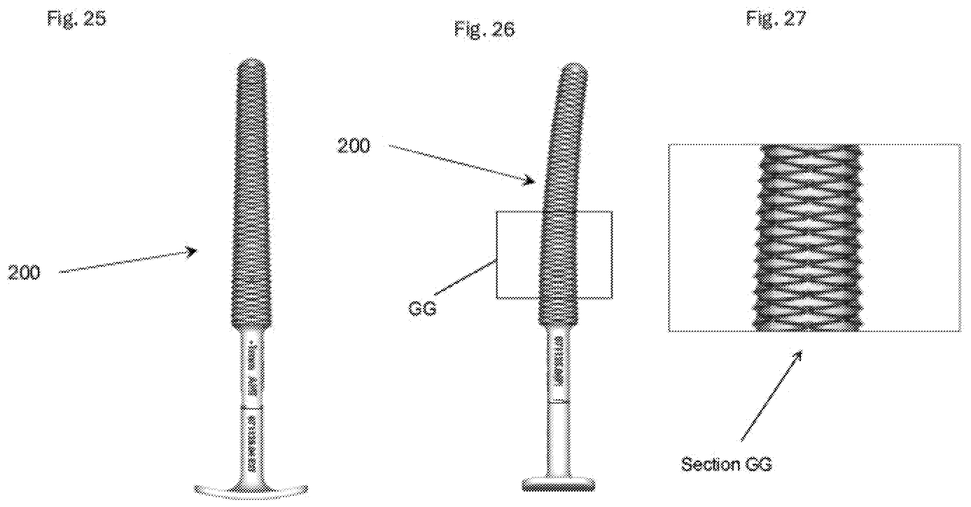

FIGS. 25-31 illustrate a customized broach 200 that is bowed to match a patient-specific anatomical curvature of the bone canal in order to bore out the canal for inserting the implant 100. The customized broach may be provided in various sizes to aid in the preparation of the bone canal starting with the smallest moving to a broach that is nominally sized to the implant. This staged broaching prepares the bone for an optimal “scratch” fit and initial engagement of the 3D porous coating with the bone.

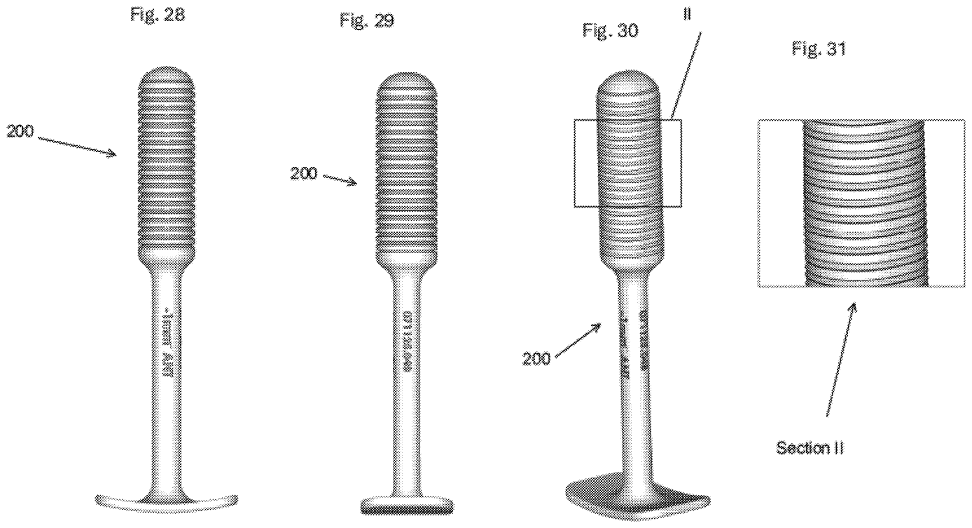

More specifically, the broach 200 shown in FIGS. 25-27 is customized for insertion into a femur, and the broach shown in FIGS. 28-31 the implant 100 that is designed for insertion into a tibia, as shown in FIGS. 14-23. These illustrate how the broach matches the implant shape and size. The tibia version of the broach has different cutting features, illustrated in detail in FIG. 23, however, the cutting effect is the same in both embodiments.

As used herein, a ‘customized’ broach refers to a broach having a geometry derived from patient-specific anatomical data, such as three-dimensional imaging of the bone canal. In some embodiments, the broach is designed using the same anatomical dataset used to design the implant, such that the broach and the implant share a corresponding three-dimensional curvature and cross-sectional profile. In this manner, preparation of the bone canal using the customized broach produces a cavity that is dimensioned relative to the implant to substantially conform to the external geometry of the implant and to enable an interference or press-fit engagement between the porous outer structure of the implant and the surrounding bone. Patient-specific anatomical data may be obtained using imaging modalities such as computed tomography (CT), magnetic resonance imaging (MRI), or other three-dimensional imaging techniques, and may be used to generate a digital model of the patient's bone canal.

It is understood that the embodiments described herein are merely illustrative of the present invention. Variations in the bone-anchored implant may be contemplated by one skilled in the art without limiting the intended scope of the invention herein disclosed.

What is claimed is:

1: A bone-anchored implant for osseointegration, comprising:
a monolithic main body extending along a longitudinal axis having a proximal end and a distal end, the main body being patient-specific and shaped to conform to a three-dimensional anatomical curvature of a bone canal derived from patient anatomical data;
the main body including a solid core and a porous outer structure integrally formed with the solid core, the porous outer structure comprising a three-dimensional lattice;
wherein the porous outer structure is configured to establish a press-fit engagement with the bone canal and promote bone ingrowth;
wherein the proximal end of the main body includes a tapered region and a rounded insertion surface configured to reduce insertion stress and stress shielding; and
wherein the distal end of the main body includes a coupling interface configured to mechanically engage a prosthetic connector assembly.

2: The implant of claim 1, wherein the main body structure being a metal structure that is manufactured using additive manufacturing.

3: The implant of claim 2, wherein the additive manufacturing process comprises 3D printing.

4: The implant of claim 2, wherein the metal is titanium, and the porous outer structure is a porous titanium coating monolithically integrated with the solid core.

5: The implant of claim 1, wherein the porous outer structure has a thickness ranging from 0.5 millimeters to 1.5 millimeters.

6: The implant of claim 1, wherein the porous outer structure has a pore size ranging from 400 microns to 800 microns.

7: The implant of claim 1, wherein the porous outer structure has a thickness of approximately 1 millimeter and a pore size of approximately 500 microns.

8: The implant of claim 1, wherein the tapered region is coated with titanium nitride.

9: The implant of claim 1, wherein the distal end of the main body has a machined smooth surface coated with titanium nitride for increased wear resistance.

10: The implant of claim 1, wherein the connector assembly comprises:
a dual cone having a proximal end configured to be received within an opening in the distal end of the main body;
a first fastener configured to secure the dual cone to the main body;
a taper sleeve and a bushing configured to be coupled to a distal end of the dual cone ; and
a second fastener configured to secure the taper sleeve and bushing to a prosthetic connector.

11: The implant of claim 10, wherein the first and second fasteners are locking screws.

12: A method of implanting a bone-anchored osseointegration implant, comprising:
obtaining patient-specific anatomical data of a bone canal;
forming an implant having:
a patient-specific geometry corresponding to the anatomical curvature of the bone canal, and
a porous outer structure comprising a lattice having a thickness between 0.5 mm and 1.5 mm and a pore size between 400 microns and 800 microns;
preparing the bone canal using a broach having a geometry corresponding to the patient-specific geometry of the implant;
inserting the implant into the prepared bone canal such that the porous outer structure interferentially engages the bone canal to create a press-fit fixation;
and securing a prosthetic connector to the implant.

13: The method of claim 12, wherein the broach used to prepare the bone canal includes a curved longitudinal profile that matches the patient-specific anatomical curvature of the implant.

14: The method of claim 12, wherein the step of inserting the implant comprises advancing the implant into the bone canal to create an interference fit between the porous outer structure and cortical bone.

15: The method of claim 12, wherein the porous outer structure has a thickness of approximately 1 mm and an average pore size of approximately 500 microns.

16: The method of claim 12, wherein securing the prosthetic connector comprises attaching a connector assembly including a dual cone and at least one locking fastener to the distal end of the implant.
